(19) United States
(12) Patent Application Publication
(10) Pub. No.: US 2025/0186025 A1
(43) Pub. Date: Jun. 12, 2025

(54) Ultrasonic Wave Probe

(71) Applicant: SEIKO EPSON CORPORATION, Tokyo (JP)

(72) Inventors: Tomohiro SAYAMA, Matsumoto (JP); Osamu MURAYAMA, Yamagata (JP); Seiji IZUO, Shiojiri (JP)

(21) Appl. No.: 18/975,539

(22) Filed: Dec. 10, 2024

(30) Foreign Application Priority Data
Dec. 11, 2023 (JP) 2023-208532

(51) Int. Cl. A61B 8/00

(52) U.S. Cl. CPC A61B 8/4444

(57) ABSTRACT

An ultrasonic wave probe includes an ultrasonic wave transmitter and receiver that transmits ultrasonic waves to a living body and that receives ultrasonic waves that were reflected inside the living body and a housing that accommodates the ultrasonic wave transmitter and receiver, wherein the housing is provided with a guide that indicates a contact angle of the ultrasonic wave transmitter and receiver corresponding to a measurement site inside the living body.

The present application is based on, and claims priority from JP Application Serial Number 2023-208532, filed Dec. 11, 2023, the disclosure of which is hereby incorporated by reference herein in its entirety.

### BACKGROUND

### 1. Technical Field

The present invention relates to an ultrasonic wave probe.

### 2. Related Art

With respect to an ultrasonic wave probe for inspecting the inside of a living body using ultrasonic waves, an ultrasonic wave probe for performing transmission and reception of ultrasonic waves is brought into contact with a predetermined position on the surface of the living body and a process is performed for transmitting and receiving ultrasonic waves to and from the living body (for example, JP-A-2003-079622).

The ultrasonic wave probe (inspection apparatus) described in JP-A-2003-079622 includes a probe section to be placed on a living body, and a positioning member provided at the tip of the probe section. The positioning member has a positioning window section, and a translucent member provided with a cross-shaped guide is mounted on the positioning window section. Also, in this inspection apparatus, the probe section is disposed so that the cross of the guide overlaps a predetermined part, such as the navel, of the living body.

By superimposing the guide of the apparatus of JP-A-2003-079622 on the predetermined part of the living body, the probe section can be arranged at the same position of the living body when ultrasonic inspection of the vicinity of the predetermined part is repeatedly performed. However, the apparatus only positions with respect to a predetermined location on the surface of a living body, and does not position with respect to the surface of predetermined tissue (for example, fat, muscle, viscera, and the like) inside the living body. That is, in inspection of a living body using ultrasonic waves, it is necessary to appropriately receive ultrasonic waves that were reflected at the boundary of the tissue in the living body, but the surface of the living body and the boundary of the tissue are not necessarily parallel to each other. Therefore, even if the probe section can be positioned at a predetermined part of the living body as in a JP-A-2003-079622, it is not always possible to transmit ultrasonic waves at an appropriate angle with respect to the tissue inside the living body, and there is a demand for a configuration in which the ultrasonic probe can be positioned at a position with higher transmission and reception efficiency.

### SUMMARY

An ultrasonic wave probe according to a first aspect of the present disclosure includes an ultrasonic wave transmitter and receiver that transmits ultrasonic waves to a living body and that receives ultrasonic waves that were reflected inside the living body and a housing that accommodates the ultrasonic wave transmitter and receiver, wherein the housing is provided with a guide that indicates a contact angle of the ultrasonic wave transmitter and receiver corresponding to a measurement site inside the living body.

### BRIEF DESCRIPTION OF THE DRAWINGS

FIG. 1 is a diagram showing schematic configuration of an ultrasonic wave device according to a first embodiment.

FIG. 2 is a flowchart showing a method of operating the ultrasonic wave device according to the present embodiment.

FIG. 3 is a diagram showing a method of specifying the contact angle of an ultrasonic wave probe (ultrasonic wave transmitter and receiver) of the present embodiment.

FIG. 4 is a diagram showing a schematic configuration of an ultrasonic wave device according to a second embodiment.

FIG. 5 is a diagram showing a schematic configuration of an ultrasonic wave probe according to a fourth modification.

### DESCRIPTION OF EMBODIMENTS

### First Embodiment

Hereinafter, a first embodiment of the present disclosure will be described.

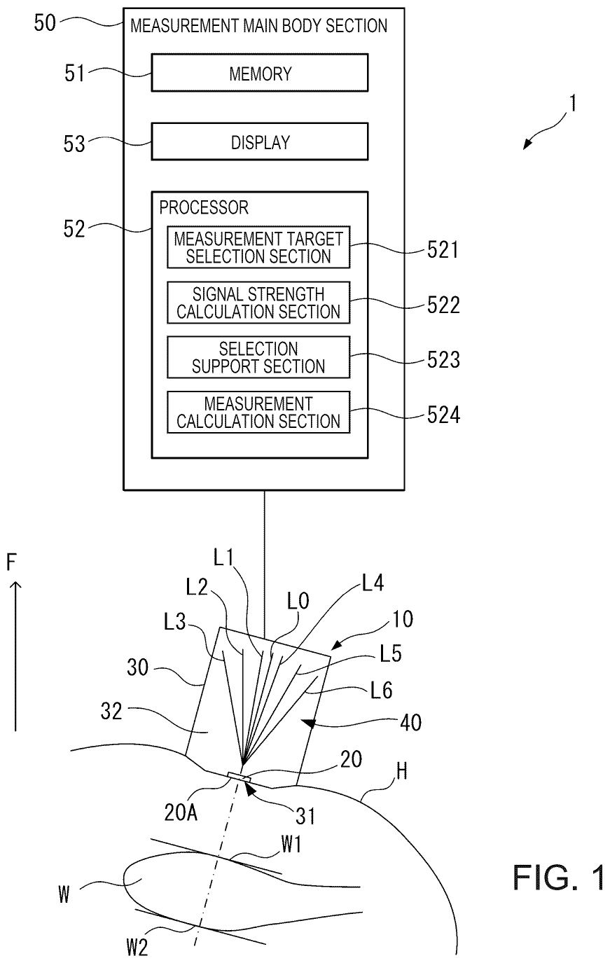

FIG. 1 is a diagram showing schematic configuration of an ultrasonic wave device 1 of the present embodiment.

The ultrasonic wave device 1 is provided with an ultrasonic wave probe 10 and a measurement main body section 50.

The ultrasonic wave probe 10 is a device for measuring ultrasonic waves by bringing the probe into contact with a living body H, such as a human body, and is provided with an ultrasonic wave transmitter and receiver 20 and a housing 30. Configuration of ultrasonic wave transmitter and receiver 20

The ultrasonic wave transmitter and receiver 20 is a transmitter and receiver that transmits ultrasonic waves to a living body and that receives ultrasonic waves that were reflected inside the living body. The ultrasonic wave transmitter and receiver 20 is not particularly limited as long as it can transmit and receive ultrasonic waves to and from a living body, for example. For example, the ultrasonic wave transmitter and receiver 20 is not particularly limited as long as it is an element capable of transmitting and receiving ultrasonic waves. For example, a bulk-type ultrasonic element may be used in which ultrasonic waves are transmitted by applying a voltage to a piezoelectric body to vibrate the piezoelectric body itself, and the reflected wave is detected by a reception signal output by the distortion of the piezoelectric body itself due to the reflected wave.

Alternatively, it may be a thin film type ultrasonic element in which a plurality of ultrasonic transducers, in which piezoelectric elements are arranged in thin film-shaped vibrating sections, are arranged in an array, and ultrasonic waves are transmitted by vibrating the vibrating sections by applying voltage to the piezoelectric elements. In such a thin film type ultrasonic element, a reception signal is output from the piezoelectric elements by the vibration films being vibrated by reflected waves. When the ultrasonic wave transmitter and receiver 20 receives a reflected wave from the living body H, it outputs a reception signal with a signal value corresponding to the sound pressure of the received ultrasonic wave. In the present embodiment, the ultrasonic wave transmitter and receiver 20 continues to receive reflected waves for a predetermined period from the transmission timing of the ultrasonic waves. Therefore, it becomes a reception signal that includes the change of the signal value along the time series output from the ultrasonic wave transmitter and receiver 20, and the reception signal is output to the measurement main body section 50.

### Configuration of Housing 30

The housing 30 houses the ultrasonic wave transmitter and receiver 20 inside. Specifically, the housing 30 is provided with a window section 31, and an ultrasonic wave transmission and reception surface 20A of the ultrasonic wave transmitter and receiver 20 is arranged on the window section 31. The window section 31 may be provided with an acoustic matching layer or the like that also functions as protection for the transmission and reception surface 20A. Although not shown, the housing 30 internally houses, among other things, a drive control circuit for controlling the operation of the ultrasonic wave transmitter and receiver 20, a communication section for communicating with the measurement main body section 50, the ultrasonic wave transmitter and receiver 20, the drive control circuit, a power supply section for supplying power to the communication section, and the like. Assuming that the direction along the normal line of the transmission and reception surface 20A of the ultrasonic wave transmitter and receiver 20 is defined as the axial direction of the housing 30, the housing 30 has a side wall 32 (exterior surface) that is elongated in the axial direction. The side wall 32 is provided with a guide 40 indicating the contact angle of the ultrasonic wave transmitter and receiver 20 at the time when the ultrasonic wave probe 10 is brought into contact with the living body H to perform ultrasonic measurement.

### Configuration of Guide 40

The guide 40 is provided on the side wall 32 of the housing 30, that is, at a position that can be easily visually checked by a user using the ultrasonic wave probe 10. As described above, the guide 40 indicates the contact angle of the ultrasonic wave transmitter and receiver 20 with respect to the living body H. In this embodiment, the position of the ultrasonic wave transmitter and receiver 20 is fixed with respect to the housing 30. Therefore, the contact angle of the ultrasonic wave transmitter and receiver 20 with respect to the living body H is determined by the contact angle of the housing 30 (that is, the ultrasonic wave probe 10 itself) with respect to the living body H. Therefore, the guide 40 instructs the contact direction of the housing 30 with respect to the living body H. More specifically, in the present embodiment, the guide 40 is constituted by a plurality of straight lines as shown in FIG. 1. These straight lines are hereinafter referred to as instruction lines Ln, and n indicates each instruction line. In the present embodiment, an example in which five instruction lines L1 to L5 are provided is shown as an example.

Here, a straight line parallel to the normal direction of the transmission and reception surface 20A of the ultrasonic wave transmitter and receiver 20 is considered to be a reference instruction line L0. The respective instruction lines Ln extend in different directions and are inclined at different angles with respect to the reference instruction line L0. The inclination angle of each instruction line In is in an angle range (absolute value) of 5 degrees or more and 25 degrees or less with respect to the reference instruction line L0 and, for example, in the example of FIG. 1, the instruction line L1 is inclined at 5 degrees with respect to the reference instruction line L0, the instruction line L2 is inclined at 15 degrees with respect to the reference instruction line L0, and the instruction line L3 is inclined at 25 degrees with respect to the reference instruction line L0. The instruction line L4 is inclined at an angle of −5 degrees with respect to the reference instruction line L0, the instruction line L5 is inclined at an angle of −15 degrees with respect to the reference instruction line L0, and the instruction line L6 is inclined at an angle of −25 degrees with respect to the reference instruction line L0. In particular, when the living body H is a human body and the rectus abdominis muscle in the abdomen is the measurement site W of the target of measurement, the boundary of the rectus abdominis muscle is inclined at an angle of about 15 degrees with respect to the surface of the living body H. Since the angle differs from person to person, the angle does not always coincide with 15 degrees, but by providing a plurality of instruction lines Ln at close-by angles, there is a high possibility that one of the instruction lines In corresponds to an inclination angle optimum for measurement. Although a detailed description will be given later, the user can confirm the optimum instruction line Ln based on the result of a provisional measurement, and thereafter, the ultrasonic wave probe 10 can be brought into contact with the living body using the same instruction line Ln as an index.

### Configuration of Measurement Main Body Section 50

The measurement main body section 50 is a computer communicably connected to the ultrasonic wave probe 10, and examples thereof include a smartphone, a tablet terminal, and a personal computer. The measurement main body section 50 instructs the ultrasonic wave probe 10 to perform ultrasonic measurement, and receives the measurement result from the ultrasonic wave probe 10. Then, based on the received measurement result, various calculation processes are performed.

Specifically, the measurement main body section 50 includes a memory 51 for storing various data, a processor 52, and a display 53 for displaying information.

The memory 51 stores various types of data and various types of programs for performing ultrasonic measurement.

By the processor 52 reading and executing the programs stored in the memory 51, it functions as a measurement target selection section 521, a signal strength calculation section 522, a selection support section 523, and a measurement calculation section 524. The measurement target selection section 521 selects a measurement site W in the living body H based on an input operation by the user. For example, a measurable measurement site such as muscle, fat, blood vessel, or the like is displayed on the display 53, and is selected by the user.

The signal strength calculation section 522 calculates the signal strength reflected at the boundary of the selected measurement site W (see FIG. 1) based on the transmission and reception result of the ultrasonic waves received from the ultrasonic wave probe 10. For example, when a muscle is selected, the signal strength of the reflected ultrasonic wave at the boundary between the muscle and the subcutaneous fat (shallow section side boundary W1) and at the boundary between the muscle and the underlying tissue (for example, viscera) (deep section side boundary W2) is calculated. The calculated signal strength may be the signal strength of both the shallow section side boundary W1 and the deep section side boundary W2, or may be the signal strength of only one of them, or may be the average signal strength of the shallow section side boundary W1 and the deep section side boundary W2. Since ultrasonic waves that are reflected inside the living body H tend to be smaller when reflected from deep portions, it is desirable to calculate the signal strength of the reflected ultrasonic wave at the deep section side boundary W2. The selection support section 523 causes the display 53 to display the signal strength of the reflected ultrasonic wave at the boundary of the measurement site W. When the user changes the contact angle of the ultrasonic wave probe 10 (ultrasonic wave transmitter and receiver 20) with respect to the living body H, the signal strength calculated by the signal strength calculation section 522 also changes according to the contact angle. The selection support section 523 displays the change in the signal strength on the display 53, so that the user can check the contact angle having the highest signal strength. At this time, it is confirmed which of the plurality of instruction lines Ln is the line located in a front direction F of the living body H. Thereafter, when performing ultrasonic measurement, the user places the ultrasonic wave probe 10 so that the confirmed instruction line Ln aligns with the front direction F of the living body H. The measurement calculation section 524 performs various measurement calculation processes based on the results of the transmission and reception of ultrasonic waves from the ultrasonic wave probe 10. For example, in this embodiment, the thickness of the selected measurement site W is measured. As the measurement calculation process, an internal tomographic image of the living body H may be displayed, or the state of a predetermined measurement site such as a blood vessel may be measured (blood pressure measurement, pulse measurement, and the like).

### Operation Method of Ultrasonic Wave Device 1

Next, a method of operating the above-described ultrasonic wave device 1 will be described.

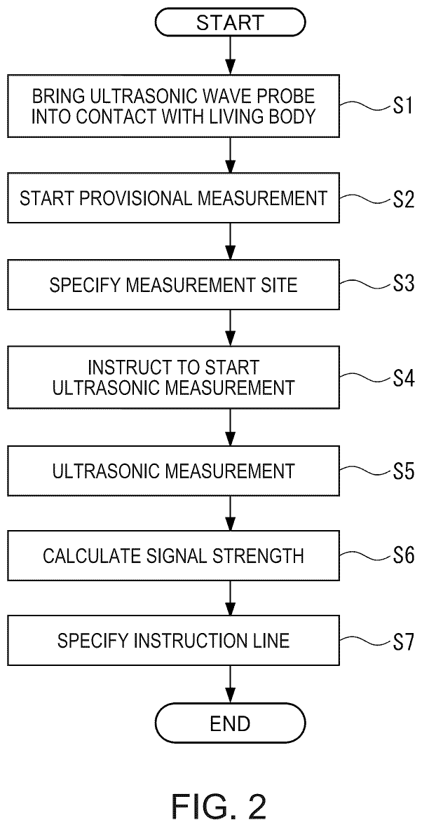

FIG. 2 is a flow chart showing an operation method of the ultrasonic wave device 1.

FIG. 2 shows the procedure of a provisional measurement when the inside of the living body H is measured for the first time using the ultrasonic wave device 1.

When the ultrasonic wave device 1 is to be used, the user brings the ultrasonic wave probe 10 into contact with the measurement position of the living body H (Step S1). For example, in a case where the rectus abdominis muscle is used as a measurement site, it is desirable that the ultrasonic wave probe 10 be brought into contact with a measurement position of about 55 mm in width and 30 mm in length from the position of the naval. The measurement position may be determined, for example, by measuring with a ruler or the like in advance, or the ultrasonic wave probe may be brought into contact with the measurement position using a positioning means of a known technique. Next, the user operates the measurement main body section 50 to execute a predetermined application relating to ultrasonic measurement, thereby instructing the start of a provisional measurement process (Step S2). By this, the measurement target selection section 521 displays a message on the display 53 prompting the selection of a measurement site. When the user selects a measurement site by operating the measurement main body section 50, the measurement target selection section 521 specifies the input measurement site (Step S3). Steps S2 and S3 may be performed before Step S1. Next, the measurement main body section 50 instructs the ultrasonic wave probe 10 to start ultrasonic measurement (Step S4). By this, the ultrasonic wave probe 10 transmits ultrasonic waves from the ultrasonic wave transmitter and receiver 20 to the inside of the living body H, receives the ultrasonic waves reflected from the inside of the living body H, and outputs a reception signal that corresponds to the received ultrasonic waves to the measurement main body section 50 (Step S5).

From the received signal, the signal strength calculation section 522 calculates the signal strength of the ultrasonic waves reflected at the boundary of the measurement site (Step S6). The selection support section 523 displays the signal strength calculated in Step S5 on the display 53. The method of displaying the signal strength is not particularly limited and, for example, the numerical value of the signal strength may be shown on the display, or a color or the like corresponding to the signal strength may be displayed on the display 53. In the case where a color corresponding to the signal strength is displayed, the user can easily determine whether or not the contact angle of the ultrasonic wave transmitter and receiver 20 is appropriate, without understanding how much signal strength is required.

As described above, the signal strength to be calculated may be the signal strength of the reflected ultrasonic wave at the shallow section side boundary W1 of the measurement site W selected by the user, the signal strength of the reflected ultrasonic wave at the deep section side boundary W2, or the sum or average signal strength of the signal strengths of both. It is more desirable to calculate the signal strength at the deep section side boundary W2 at a deep depth, from which reception of the ultrasonic wave is difficult The processes from Step S5 to Step S6 are carried out continuously. Therefore, when the user changes the contact angle of the ultrasonic wave probe 10 with respect to the living body H, then in Step S5, a reception signal corresponding to the contact angle is output to the measurement main body section 50, and the signal strength displayed on the display 53 is updated. Therefore, by changing the contact angle of the ultrasonic wave probe 10 (ultrasonic wave transmitter and receiver 20) with respect to the living body H while referring to the display 53, the user can easily confirm the contact angle at which the signal strength becomes maximum.

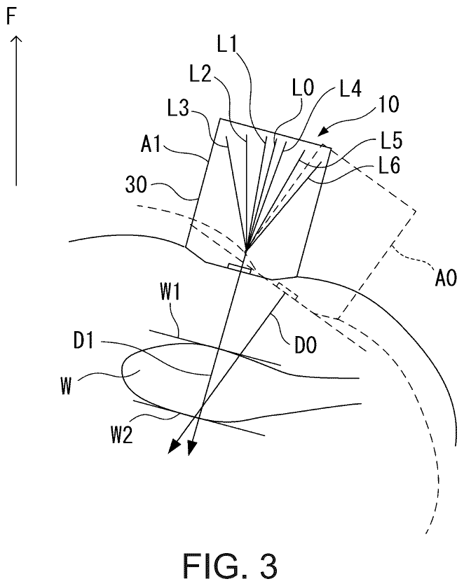

When the user specifies the contact angle at which the signal strength is maximum, the user specifies the instruction line Ln that is parallel to the front direction F of the living body H (Step S7). FIG. 3 is a diagram showing a method of specifying the contact angle of the ultrasonic wave probe 10 (ultrasonic wave transmitter and receiver 20) of the present embodiment.

In the related art ultrasonic wave probe fixing method, the ultrasonic wave probe 10 is brought into contact with the body surface of the living body H so that the reference instruction line L0, that is, the sonic axial direction of the ultrasonic wave transmitted from the ultrasonic wave transmitter and receiver 20, is perpendicular to the body surface (for example, the ultrasonic wave probe 10 in the orientation A0 in FIG. 3).

The boundary between the body surface and the measurement site W (for example, the rectus abdominis muscle) in the living body H is not necessarily parallel, or rather, is usually not parallel. Therefore, when the transmission and reception surface 20A of the ultrasonic wave transmitter and receiver 20 is placed along the body surface of the living body H as in the orientation A0, the boundary between the sonic axial direction D0 and the measurement site W is not perpendicular. In this case, the component of the reflected ultrasonic wave that is specularly reflected at the boundary of the measurement site W becomes small.

On the other hand, when the ultrasonic wave probe 10 (ultrasonic wave transmitter and receiver 20) is tilted to the orientation A1, the sonic axial direction D1 approaches a perpendicular relationship with the boundary of the measurement site W. By this, the signal strength of the reception signal obtained by receiving the ultrasonic wave reflected at the boundary of the measurement site W increases. By performing ultrasonic measurement while maintaining the orientation of the ultrasonic wave probe 10, the boundary of the measurement site W can be detected with higher accuracy, and the measurement accuracy can be improved.

In this state, among the plurality of instruction lines Ln, the instruction line Ln that is parallel to the front direction F of the living body H is specified. For example, in the case of FIG. 3, the instruction line L2 is specified as the instruction line suitable for the measurement site W. By specifying the instruction line In using the provisional measurement process described above, the contact angle of the ultrasonic wave probe 10 can be easily set on the basis of the instruction line Ln specified in Step S7 when the ultrasonic measurement is performed subsequently.

For example, in this embodiment, the contact angle of the ultrasonic wave probe 10 is set so that the instruction line Ln specified in Step S7 is parallel to the front direction F of the living body H. By this, for example, when ultrasonic measurement is periodically performed on the same measurement site W, the contact angle of the ultrasonic wave probe 10 can be kept constant, and the change in the measurement site W over time can be suitably measured. Note that in order to illustrate the measurement of the rectus abdominis muscle, an example is described in which the contact orientation of the ultrasonic wave probe 10 is maintained so that the front direction F of the living body H and the instruction line Ln specified in Step S7 are parallel to each other, but this is not a limitation. In the case where a specific target exists in a direction other than the front direction F, when the instruction line Ln is specified in Step S7, the instruction line Ln in the direction in which the target exists may be specified. For example, in the case where the side of the arm is set as the measurement position, the instruction line Ln in the direction of the side of the arm (for example, the right-hand direction for the right arm) may be specified. When ultrasonic measurement is performed at a predetermined position in a room, an instruction line Ln in the direction of a fixed object (such as an interior decoration) existing around the predetermined position may be specified.

### Effects of the Present Embodiment

The ultrasonic wave probe 10 of the first embodiment is provided with the ultrasonic wave transmitter and receiver 20 and the housing 30 for housing the ultrasonic wave transmitter and receiver 20. The ultrasonic wave transmitter and receiver 20 transmits ultrasonic waves to the living body H and receives ultrasonic waves that were reflected inside the living body H. The housing 30 is provided with a guide 40 for indicating the contact angle of the ultrasonic wave transmitter and receiver 20 corresponding to a predetermined measurement site inside the living body H.

Therefore, the user brings the ultrasonic wave probe 10 into contact with the living body H at the contact angle instructed by the guide 40 while visually checking the guide 40, so that the ultrasonic wave transmitter and receiver 20 has a contact angle corresponding to the measurement site. By this, compared with the case where the ultrasonic wave probe 10 is brought into contact with the living body H so that the transmission and reception surface 20A of the ultrasonic wave transmitter and receiver 20 is simply along the body surface of the living body H, the transmission and reception surface 20A is placed at an angle corresponding to the measurement site W, so that highly accurate ultrasonic transmission and reception to and from the measurement site W can be performed, and various measurements on the measurement site W can be performed with high accuracy. In the present embodiment, the guide 40 is an instruction line Ln (straight line) indicated on the side wall 32 (exterior surface) of the housing 30.

Therefore, the user only has to bring the ultrasonic wave probe 10 into contact with the living body H so that the direction of the instruction line Ln becomes a predetermined direction (for example, the front direction F), and the operation of placing the ultrasonic wave probe 10 on the living body H becomes easy. In this embodiment, a plurality of instruction lines Ln are provided, and the instruction lines Ln extend in different directions.

By this, the user can select one of the plurality of instruction lines Ln and bring the ultrasonic wave probe 10 into contact with the living body H using the selected instruction line Ln. That is, the user can appropriately select the instruction line Ln corresponding to each individual living body to be measured or the instruction line Ln corresponding to the type of the measurement site W. In the present embodiment, the plurality of instruction lines Ln include a reference line (reference instruction line L0) that is orthogonal to the transmission and reception surface 20A of the ultrasonic wave transmitter and receiver 20, and the other instruction lines Ln are inclined at different angles with respect to the reference instruction line L0.

By this, since the reference line (reference instruction line L0), which is the normal direction of the transmission and reception surface 20A, is provided, the user can easily grasp which of the instruction lines Ln corresponds to the appropriate contact angle. In this embodiment, the instruction lines Ln are provided within an angle range of 5 degrees or more and 25 degrees or less with respect to the reference instruction line L0.

With respect to the body surface of the living body H, the boundary of the muscle tissue, particularly the rectus abdominis muscle, in the living body H is inclined at an angle of about 15 degrees, although there are individual differences. Therefore, by providing a plurality of instruction lines In within a range of 5degrees or more and 25 degrees or less, there is a high possibility that one of these instruction lines Ln instructs an inclination angle corresponding to the boundary of the muscle tissue. By this, the user can find the instruction line Ln corresponding to his or her own measurement site.

### Second Embodiment

The first embodiment is an example of the ultrasonic wave probe 10 in which the orientation of the ultrasonic wave transmitter and receiver 20 is fixed with respect to the housing 30. On the other hand, an example of an ultrasonic wave probe in which the orientation of the ultrasonic wave transmitter and receiver 20 can be changed with respect to the housing 30 will be described as a second embodiment.

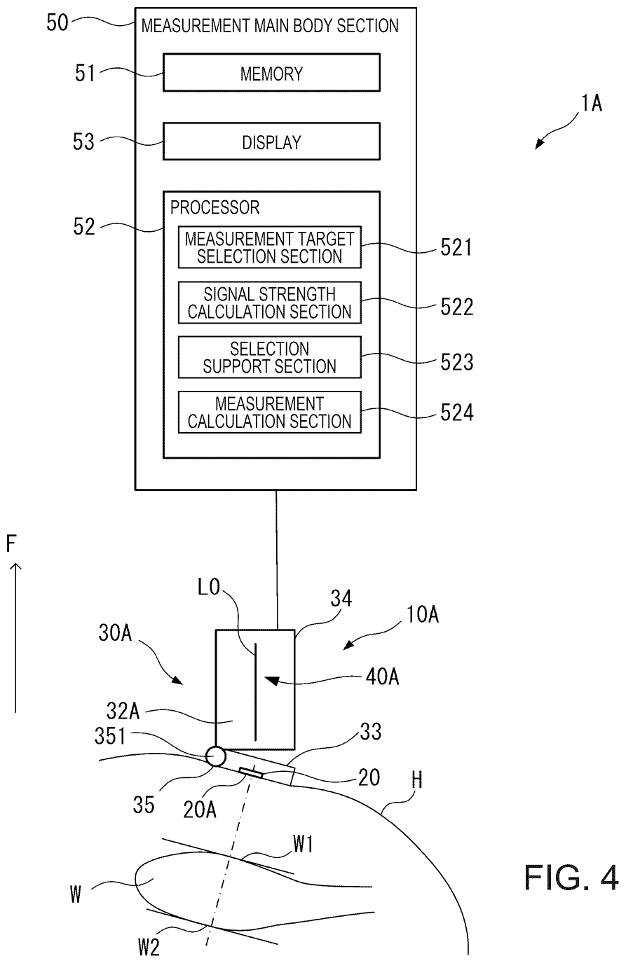

In the following description, the same reference numerals are given to the matters already described, and the description thereof is omitted or simplified. FIG. 4 is a diagram showing a schematic configuration of ultrasonic wave device 1A according to the second embodiment.

The ultrasonic wave device 1A of this embodiment has an ultrasonic wave probe 10A and a measurement main body section 50. As shown in FIG. 4, the ultrasonic wave probe 10A includes an ultrasonic wave transmitter and receiver 20 and a housing 30A. As shown in FIG. 4, the housing 30A of the present embodiment includes a head 33, a grip 34, and a hinge section 35.

The head 33 is a portion in which the ultrasonic wave transmitter and receiver 20 is housed. As in the first embodiment, the head 33 is provided with a window section 31, and the transmission and reception surface 20A of the ultrasonic wave transmitter and receiver 20 is exposed through the window section 31. Alternatively, the window section 31 may be provided with an acoustic matching layer. The grip 34 is a portion to be hand held when the user operates the ultrasonic wave probe 10A. A guide 40A is provided on the exterior surface (side wall 32A) of the grip 34. The hinge section 35 functions as a hinge for connecting the head 33 and the grip 34. The hinge section 35 further has a lock section 351 that supports the head 33 so that the head 33 can be pivoted with respect to the grip 34 and that also locks the pivot of the head 33 with respect to the grip 34. That is, in a state in which the lock section 351 is released (unlocked state), the head 33 becomes pivotable with respect to the grip 34, and in a state in which the lock section 351 is locked (locked state), the pivoting of the head 33 with respect to the grip 34 is restricted. In the present embodiment, the guide 40A is constituted by one reference instruction line L0 provided on the side wall 32A of the grip 34. In this embodiment, the grip 34 has a longitudinal direction, and a reference instruction line L0 is provided along the longitudinal direction.

How to operate ultrasonic wave device 1A Next, a method of operating the above-described

ultrasonic wave device 1A will be described. In the ultrasonic wave device 1A of the present embodiment, the provisional measurement treatment is performed by a method substantially similar to that of the first embodiment.

That is, in Step S1, the user brings the ultrasonic wave probe 10A into contact with the measurement position of the living body H. At this time, the lock section 351 releases the lock of the hinge section 35 so that the head 33 can be pivoted with respect to the grip 34. In the present embodiment, the ultrasonic wave probe 10A is brought into contact with the living body H so that the reference instruction line L0 is directed in a predetermined direction. For example, the ultrasonic wave probe 10A is brought into contact with the living body H so that the front direction F of the living body H and the reference instruction line L0 are parallel to each other. Next, in Step S2, the user operates the measurement main body section 50 to execute a predetermined application relating to the ultrasonic measurement, thereby instructing the start of the provisional measurement process. In Step S3, the user operates the measurement main body section 50 to select the measurement site W, and the measurement target selection section 521 specifies the input measurement site W. Thereafter, Step S4 to Step S6 are performed, the ultrasonic measurement is performed by the ultrasonic wave probe 10A, the signal strength of the ultrasonic waves reflected at the boundary of the measurement site W is calculated from the received signal, and the signal strength is displayed on the display 53.

Similarly to the first embodiment, the processes from Step S5 to Step S6 are continuously performed.

In this embodiment, the user changes the pivot angle of the head 33 with respect to the grip 34, and brings the ultrasonic wave probe 10A into contact with the same measurement position. Also in this case, the ultrasonic wave probe 10A is brought into contact with the living body H so that the reference instruction line L0 faces a predetermined direction, for example, so that the front direction F of the living body H and the reference instruction line L0 are parallel to each other. By changing the pivot angle of the head 33 with respect to the grip 34 in this way, as in the first embodiment, a reception signal corresponding to the contact angle of the ultrasonic wave transmitter and receiver 20 with respect to the living body H is output to the measurement main body section 50, and the signal strength displayed on the display 53 is updated. Therefore, in this embodiment, instead of Step S7, the user confirms the pivot angle of the head 33 at which the signal strength becomes maximum while referring to the display 53.

When the user specifies the contact angle (pivot angle of the head 33) at which the signal strength is maximum, the hinge section 35 is placed into a locked state by the lock section 351. By this, when the ultrasonic measurement is performed subsequently, the appropriate ultrasonic measurement can be performed by merely bringing the ultrasonic wave probe 10A into contact with the measurement position.

### Effects of the Present Embodiment

According to this embodiment, the same effects as those of the first embodiment can be obtained and, furthermore, the following effects can be obtained.

In the ultrasonic wave probe 10A of the present embodiment, the housing 30A is provided with the head 33 in which the ultrasonic wave transmitter and receiver 20 is provided, the grip 34 that supports the head 33 and that is gripped by a user, and the hinge section 35 (hinge) that connects the head 33 and the grip 34 together and that can change inclination angle of the head 33 with respect to the grip 34. The hinge section 35 is provided with the lock section 351, and can be switched between an unlocked state in which the tilt angle of the head 33 with respect to the grip 34 can be changed and a locked state in which change of the tilt angle of the head 33 with respect to the grip 34 is restricted. The guide 40 is a reference instruction line L0 displayed on the side wall 32A (exterior surface) of the grip 34. By this, in this embodiment, if the pivot angle (inclination angle) of the head 33 with respect to the grip 34 is set to a predetermined angle corresponding to the measurement site of the living body H, appropriate ultrasonic measurement can be performed by bringing the ultrasonic wave probe 10A into contact with the living body H so that the reference instruction line L0 is directed in a predetermined direction (for example, parallel to the front direction F). Therefore, unlike the first embodiment, it is not necessary to store the instruction line Ln specified by the user in Step S7, and the ultrasonic wave probe 10A can be more easily installed in the living body H in the next and subsequent ultrasonic measurement.

### MODIFICATIONS

It should be noted that the present disclosure is not limited to the above-described embodiments, and the present disclosure includes modifications, improvements, and configurations obtained by appropriately combining the embodiments within a range in which the object of the present disclosure can be achieved.

### First Modification

In the second embodiment, the user manually changes the pivot angle between the head 33 and the grip 34 and manually changes the locked state of the hinge section 35. In contrast to this, the hinge section 35 of the ultrasonic wave probe 10A may be provided with a pivot drive section, such as a motor, so that the angle between the head 33 and the grip 34 can be automatically changed.

In the provisional measurement process, the pivot angle between the head 33 and the grip 34 may be pivotably scanned to search for an angle at which the signal strength of the received signal, which is based on the ultrasonic wave reflected at the boundary of the measurement site, is maximized. With such a configuration, it is possible to automatically set the contact angle of the ultrasonic wave transmitter and receiver 20 with respect to the living body H to an optimum angle without requiring the user to check the display 53. By storing the searched pivot angle in a storage device, such as the memory 51, the pivot driving section need only be controlled by reading out the angle in subsequent ultrasonic wave measurements. In this case, the user merely has to bring the ultrasonic wave probe 10A into contact with the living body H so that the reference instruction line L0 indicated by the guide 40 is parallel to a predetermined direction (for example, the front direction F of the living body H), whereby the contact angle of the ultrasonic wave transmitter and receiver 20 is automatically controlled, and ultrasonic measurement on the measurement site can be appropriately performed.

### Second Modification

In the first embodiment, a plurality of instruction lines Ln provided on the side wall 32 of the housing 30 are exemplified as the guide 40, but this is not a limitation. Any configuration may be adopted as long as the user can easily check the contact direction of the ultrasonic wave probe 10 and, for example, the direction may be indicated by providing a protruding point or a recess.

Alternatively, the side wall 32 may be provided with an instruction display. In the configuration in which the instruction line is shown on the instruction display, when the user specifies the instruction line Ln corresponding to the maximum signal strength in Step S7, only the instruction line Ln may be displayed thereafter, and the other instruction lines In may be hidden until the provisional measurement process is performed next.

Although the example has been described in Step S6 in which the calculated signal strength or a color corresponding to the signal strength is displayed on the display 53, the calculated signal strength or the color corresponding to the signal strength may be displayed on an instruction display when the ultrasonic wave probe 10 is provided with an instruction display.

### Third Modification

In the second embodiment, the hinge section 35 is provided at one end of the head 33 in one direction and connected to the grip 34, but the hinge section 35 may be provided at the center of the head 33 in one direction. In this case, the ultrasonic wave transmitter and receiver 20 of the head 33 can be pivoted in the clockwise and counterclockwise directions from a position perpendicular to the reference instruction line L0.

### Fourth Modification

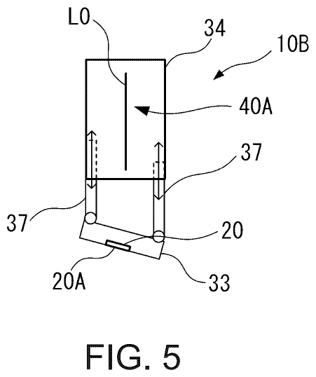

In the second embodiment, an example was shown in which the hinge of the present disclosure is constituted by hinge section 35, but this is not a limitation. FIG. 5 is a diagram showing a schematic configuration of an ultrasonic wave probe 10B according to a fourth modifcation. For example, as shown in FIG. 5, leg sections 37 may be provided on both sides in one direction of the head 33 and configured capable of advancing and retreating with respect to the grip 34 In this case, each leg section 37 is provided with a lock mechanism for restricting forward and backward movement.

With such a configuration, the inclination angle of the head 33 with respect to the grip 34 can be controlled by controlling the advancing and retreating distance of each of the leg sections 37.

### SUMMARY OF DISCLOSURE

An ultrasonic wave probe according to a first aspect of the present disclosure includes an ultrasonic wave transmitter and receiver that transmits ultrasonic waves to a living body and that receives ultrasonic waves that were reflected inside the living body and a housing that accommodates the ultrasonic wave transmitter and receiver, wherein the housing is provided with a guide that indicates a contact angle of the ultrasonic wave transmitter and receiver corresponding to a measurement site inside the living body.

According to this aspect, the user brings the ultrasonic wave probe into contact with the living body at the contact angle instructed by the guide while visually confirming the guide, so that the ultrasonic wave transmitter and receiver has a contact angle corresponding to the measurement site. By this, compared with the case where the transmission and reception surface of the ultrasonic wave transmitter and receiver is simply placed along the body surface of the living body, the transmission and reception surface is installed at an angle corresponding to the measurement site, and therefore, highly accurate ultrasonic transmission and reception to and from the measurement site can be performed, and various measurements on the measurement site can be performed with high accuracy.

The ultrasonic wave probe of the present aspect may be such that the guide is a straight line shown on an exterior surface of the housing.

By this, the user need only bring the ultrasonic wave probe into contact with the living body so that the elongated direction of the straight line becomes a predetermined direction (for example, the front direction of the living body) and the setting operation of the ultrasonic wave probe to the living body becomes easy.

The ultrasonic wave probe of the present aspect may be such that a plurality of the straight lines are provided and the straight lines extend in different directions.

By this, the user can select one of the straight lines and bring the ultrasonic wave probe into contact with the living body using the selected straight line. By selecting an optimum straight line for each measurement point in the living body or each measurement site in the living body, the ultrasonic wave probe can be brought into contact with the living body at an optimum contact angle corresponding to each target of measurement.

The ultrasonic wave probe of the present aspect may be such that a reference line is included that is orthogonal to a transmission and reception surface of the ultrasonic waves of the ultrasonic wave transmitter and receiver and the other straight lines are inclined at different angles with respect to the reference line.

By providing the reference line as the normal direction of the transmission and reception surface, the user can easily grasp which straight line of the straight lines should be selected to bring the ultrasonic wave probe into contact with the living body.

The ultrasonic wave probe of the present aspect may be such that the straight lines are provided within an angle range of 5 degrees or more and 25 degrees or less with respect to the reference line.

In general, muscle tissue in a living body, in particular, the boundary of rectus abdominis muscles, are at a tilt of about 15 degrees with respect to the surface of the living body. Therefore, by providing a plurality of instruction lines Ln within a range of 5 degrees or more and 25 degrees or less, there is a high possibility that one of these instruction lines Ln instructs an inclination angle corresponding to the boundary of the muscle tissue. By this, the user can find the instruction line Ln corresponding to the measurement site of the user.

The ultrasonic wave probe of the present aspect may be such that the housing includes a head provided with the ultrasonic wave transmitter and receiver, a grip that supports the head and that is gripped by a user, and a hinge that connects the head and the grip together and that is configured to enable change of an inclination angle of the head with respect to the grip, wherein the hinge is switchable between an unlocked state in which the inclination angle of the head with respect to the grip is changable and a locked state in which change of the inclination angle of the head with respect to the grip is restricted and the guide is a straight line displayed on the exterior surface of the grip.

With such a configuration, the ultrasonic wave probe is brought into contact with the living body so that the straight line of the guide faces a predetermined direction. In the unlocked state, the tilt angle of the head with respect to the grip is set so that the ultrasonic wave transmitter and receiver has a predetermined angle corresponding to the measurement site of the living body. Then afterward, the grip is placed into the locked state. By this, when a subsequent ultrasonic measurement is carried out, the ultrasonic wave probe can be brought into contact with the living body so that the ultrasonic wave transmitter and receiver has an appropriate inclination angle with respect to the measurement site by merely bringing the ultrasonic wave probe into contact with the living body so that the straight line of the guide is directed in a predetermined direction with respect to the living body, without changing the inclination angle of the head with respect to the grip.

What is claimed is:

1. An ultrasonic wave probe comprising:
an ultrasonic wave transmitter and receiver that transmits ultrasonic waves to a living body and that receives ultrasonic waves that were reflected inside the living body and
a housing that accommodates the ultrasonic wave transmitter and receiver, wherein
the housing is provided with a guide that indicates a contact angle of the ultrasonic wave transmitter and receiver corresponding to a measurement site inside the living body.

2. The ultrasonic wave probe according to claim 1, wherein
the guide is a straight line shown on an exterior surface of the housing.

3. The ultrasonic wave probe according to claim 2, wherein
a plurality of the straight lines are provided and
the straight lines extend in different directions.

4. The ultrasonic wave probe according to claim 3, wherein
the straight lines include a reference line that is orthogonal to an ultrasonic wave transmission and reception surface of the ultrasonic wave transmitter and receiver, and others of the straight lines are inclined at different angles with respect to the reference line.

5. The ultrasonic wave probe according to claim 4, wherein
the straight lines are provided within an angle range of 5 degrees or more and 25 degrees or less with respect to the reference line.

6. The ultrasonic wave probe according to claim 1, wherein
the housing includes
a head provided with the ultrasonic wave transmitter and receiver,
a grip that supports the head and that is gripped by a user, and
a hinge that connects the head and the grip together and that is configured to enable change of an inclination angle of the head with respect to the grip, wherein
the hinge is switchable between an unlocked state in which the inclination angle of the head with respect to the grip is changeable and a locked state in which change of the inclination angle of the head with respect to the grip is restricted and
the guide is a straight line displayed on the exterior surface of the grip.
